(19) United States
(12) Patent Application Publication
(10) Pub. No.: US 2003/0085070 A1
(43) Pub. Date: May 8, 2003

(54) Waterproof earphone

(76) Inventor: Timothy K. Wickstrom, Elk Grove Village, IL (US)

Correspondence Address: Thomas K. Stine, Wallenstein & Wagner, Ltd., 53rd Floor, 311 South Wacker Drive, Chicago, IL, 60606-6630, US

(21) Appl. No.: 09/986,528

(22) Filed: Nov. 7, 2001

(51) Int. Cl. A61B 7/02

(52) U.S. Cl. 181/135; 181/130

(57) ABSTRACT

A waterproof earphone is disclosed. The earphone comprises a housing having a first end, a second end, and a housing bore extending there between. The housing is dimensioned for insertion into a human ear canal. The earphone further includes a sleeve having a first end and a second end, and an axial bore extending there between. The sleeve is disposed within the housing. The earphone still further includes a receiver disposed within the sleeve's axial bore. The receiver has a cable extending outwardly past the first sleeve end. An end-cap has first and second ends, and the end-cap first end matingly engages the housing's second end. A waterproof membrane seals the end-cap's second end.

### RELATED APPLICATIONS

Not Applicable.

### FEDERALLY SPONSORED RESEARCH OR DEVELOPMENT

Not Applicable.

### TECHNICAL FIELD

The present invention relates to a waterproof earphone.

### BACKGROUND OF THE INVENTION

There are times when an earphone is needed in an extremely wet environment, such as in an ocean rescue situation when a rescuer must jump out of a helicopter into the ocean. For effective use, the earphone must be both comfortable, as well as capable of keeping out water.

The present invention is provided to solve these and other problems.

### SUMMARY OF THE INVENTION

It is an object of the invention to provide a waterproof earphone.

In accordance with the invention, the earphone comprises a housing having a first end, a second end, and a housing bore extending there between. The housing is dimensioned for insertion into a human ear canal. The earphone further comprises a sleeve having a first end and a second end, and an axial bore extending there between. The sleeve is disposed within the housing. The earphone still further comprises a receiver disposed within the sleeve's axial bore. The receiver has a cable extending outwardly past the first sleeve end. An end-cap has first and second ends. The end-cap first end matingly engages the housing's second end. A waterproof membrane seals the end-cap's second end.

It is contemplated that the housing is resilient, such as made of foam rubber.

It is further contemplated that an o-ring seal is disposed between the sleeve and the end-cap.

It is still further contemplated that the first sleeve end is potted, such as with an epoxy, about the cable.

It is yet further contemplated that the end-cap first end surrounds the sleeve second end, and that the first sleeve end is flanged and the flanged first end engages the housing's first end.

These and other aspects and attributes of the present invention will be discussed with reference to the following drawings and accompanying specification.

### BRIEF DESCRIPTION OF THE DRAWINGS

FIG. 1 is a sectional drawing of an earphone in accordance with the invention.

### DETAILED DESCRIPTION OF THE PREFERRED EMBODIMENT

While this invention is susceptible of embodiment in many different forms, there is shown in the drawings and will herein be described in detail a preferred embodiment of the invention with the understanding that the present disclosure is to be considered as an exemplification of the principles of the invention and is not intended to limit the broad aspect of the invention to the embodiment illustrated.

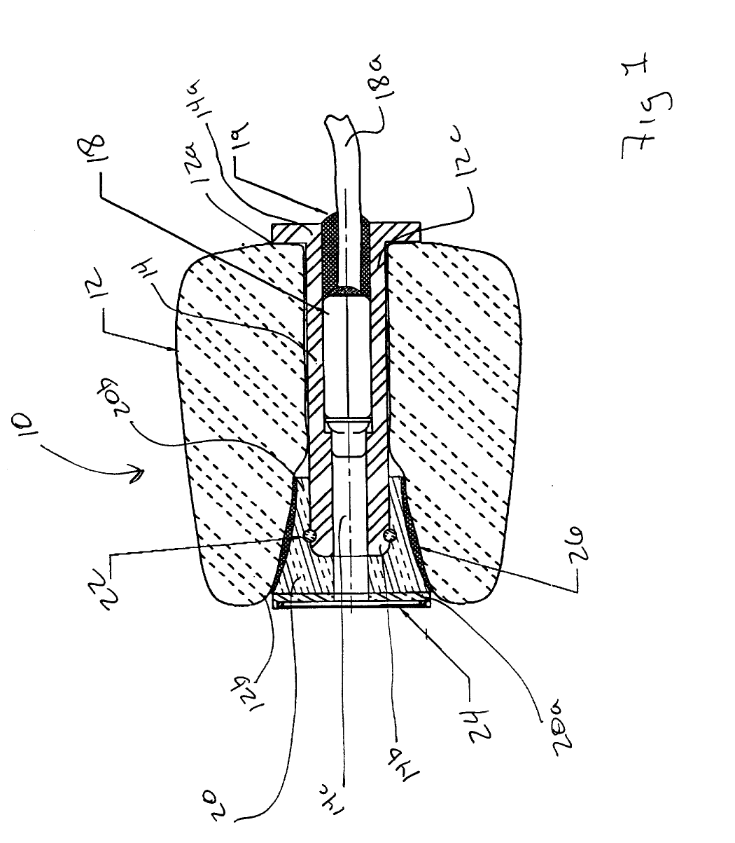

A waterproof earphone 10 according to the invention is illustrated in FIG. 1. The earphone 10 comprises a resilient housing 12 having a first end 12a, a second end 12b, and a housing bore 12c extending there between. The housing 12 is dimensioned for insertion into a human ear canal (not shown). The housing is preferably made of foam rubber.

The earphone 10 further comprises a rigid sleeve 14 having a first end 14a and a second end 14b, and an axial bore 14c extending there between. The sleeve 14 is disposed within the housing bore 12c. The sleeve 14 is retained in the housing bore 12c by an interference fit. The first sleeve end 14a is flanged and the flanged first sleeve end 14a engages the housing's first end 12a.

A receiver 18, such as a Knowles FK receiver, is disposed within the sleeve's axial bore 14c. The Knowles FK receiver is available from Knowles Electronics LLC, of Itasca Ill., the assignee of the present application. The receiver 18 has a cable 18a extending outwardly past the first sleeve end 14a. The first sleeve end is potted about the cable 18a with an epoxy 19.

A rigid end-cap 20 has first and second ends 20a, 20b. The end-cap first end 20a engages the housing's second end 12b. An o-ring seal 22 is disposed between the sleeve 14 and the end-cap 20. A waterproof membrane 24 seals the end-cap's second end 20b.

The sleeve 14 and the end-cap 20 are formed of plastic. An adhesive 26 secures the end-cap to the housing 12.

While the specific embodiments have been illustrated and described, numerous modifications come to mind without significantly departing from the spirit of the invention and the scope of protection is only limited by the scope of the accompanying claims.

I claim:

1. A waterproof earphone comprising:
a housing having a first end, a second end, and a housing bore extending there between, the housing dimensioned for insertion into a human ear canal;
a sleeve having a first end and a second end, and an axial bore extending there between, wherein the sleeve is disposed within the housing;
a receiver disposed within the sleeve's axial bore, wherein the receiver has a cable extending outwardly past the first sleeve end;
an end-cap having first and second ends, wherein the end-cap first end matingly engages the housing's second end; and
a waterproof membrane sealing the end-cap's second end.

2. The earphone of claim 1 wherein the housing is resilient.

3. The earphone of claim 2 wherein the housing is made of foam rubber.

4. The earphone of claim 1 including an o-ring seal between the sleeve and the end-cap.

5. The earphone of claim 1 wherein the first sleeve end is potted about the cable.

6. The earphone of claim 5 wherein the first sleeve end is potted about the cable with an epoxy.

7. The earphone of claim 1 wherein the end-cap first end surrounds the sleeve second end.

8. The earphone of claim 1 wherein the first sleeve end is flanged, and the flanged first end engages the housing's first end.

9. The earphone of claim 1 wherein the sleeve is rigid.

10. The earphone of claim 9 wherein the sleeve is formed of plastic.

11. The earphone of claim 1 including an adhesive securing the end-cap to the housing.

12. The earphone of claim 1 wherein the end-cap is rigid.

13. The earphone of claim 12 wherein the end-cap is plastic.

14. A waterproof earphone comprising:
a resilient housing having a first end, a second end, and a housing bore extending there between, the housing dimensioned for insertion into a human ear canal;
a rigid sleeve having a first end and a second end, and an axial bore extending there between, wherein the sleeve is disposed within the housing;
a receiver disposed within the sleeve's axial bore, wherein the receiver has a cable extending outwardly past the first sleeve end;
an end-cap having first and second ends, wherein the end-cap first end matingly engages the housing's second end; and
a waterproof membrane sealing the end-cap's second end.

15. The earphone of claim 14 wherein the housing is made of foam rubber.

16. The earphone of claim 14 including an o-ring seal between the sleeve and the end-cap.

17. The earphone of claim 14 wherein the first sleeve end is potted about the cable.

18. The earphone of claim 15 wherein the first sleeve end is potted about the cable with an epoxy.

19. The earphone of claim 14 wherein the end-cap first end surrounds the sleeve second end.

20. The earphone of claim 14 wherein the first sleeve end is flanged and the flanged first end engages the housing's first end.

21. The earphone of claim 14 wherein the sleeve is formed of plastic.

22. The earphone of claim 14 including an adhesive securing the end-cap to the housing.

23. A waterproof earphone comprising:
a resilient housing having an axial bore, wherein the housing is dimensioned for insertion into a human ear canal;
a rigid sleeve disposed within the housing bore and having a first end and a second end; and
a receiver disposed within the sleeve, the receiver having a cable extending outwardly from the sleeve's first end and a sound port for generating sound to be transmitted outwardly from the sleeve's second end.

24. The receiver of claim 23 including an end-cap disposed adjacent the sleeve's second end.

25. The receiver of claim 24 wherein the end-cap includes a waterproof membrane.

26. The receiver of claim 25 wherein the end-cap surrounds the sleeve's second end.
